(19) United States
(12) Patent Application Publication
(10) Pub. No.: US 2004/0002592 A1
(43) Pub. Date: Jan. 1, 2004

(54) Detection of rare Candida species

(76) Inventors: Hermann Einsele, Tubingen (DE); Jurgen Loffler, Reutlingen (DE)

Correspondence Address: KNOBBE MARTENS OLSON & BEAR LLP, 2040 MAIN STREET, FOURTEENTH FLOOR, IRVINE, CA, 92614, US

(21) Appl. No.: 10/426,159

(22) Filed: Apr. 29, 2003

Related U.S. Application Data
(63) Continuation of application No. PCT/EP01/11805, filed on Oct. 12, 2001.

(30) Foreign Application Priority Data
Oct. 30, 2000 (DE) DE 100 53 821.5

(51) Int. Cl. C07H 21/02; C07H 21/04

(52) U.S. Cl. 536/23.1

(57) ABSTRACT

The present invention relates to a nucleic acid molecule, to a kit which comprises the nucleic acid molecule, to methods for detecting fungi in clinical material and for determining the sequence of ribosomal fungal genes, and to kits for carrying out these methods.

### CROSS REFERENCES TO RELATED APPLICATIONS

This application is a continuation of the International Patent Application PCT/EP01/11805, filed Oct. 12, 2001, and designating the U.S., published in German, which claims priority to a German patent application DE 100 53 821.5, filed Oct. 30, 2000, both of which are incorporated herein by reference in their entireties.

### BACKGROUND OF THE INVENTION

1. Field of the Invention

The present invention relates to a nucleic acid molecule, to a kit which comprises the nucleic acid molecule, to a method for detecting fungi in clinical material and for determining the sequence of ribosomal fungal genes, and to kits for carrying out these methods.

2. Description of the Related Art

Nucleic acid molecules used for detecting fungi and for determining the sequence of ribosomal fungal genes, and methods for detecting fungi in clinical material are known from various publications.

The problem of fungal infections has assumed considerable proportions in the last 20 years. This is attributable in particular to the increase in patients with weakened immune defenses, intensive immunosuppressant chemotherapies, the increasing use of broad-spectrum antibiotics and of central venous catheters. Important mycoses, i.e. the infectious diseases caused by fungi, are, for example, candidal mycosis, aspergillus mycosis or mucor mycosis.

Invasive candidiasis as an example of candidal mycosis is a life-threatening infection in immunocompromised hosts such as, for example, recipients of bone marrow or organ transplants, in patients with extensive chemotherapy and in AIDS patients. In addition, systemic candidal infections are observed in patients after extensive surgical operations or after burns, associated with intensive antibiotic therapies, indwelling catheters, patients with diabetes mellitus and in elderly patients; concerning this, see Wenzel, R. P. 1995, “Nosocomial candidemia: risk factors and attributable mortality”, Clin. Infect. Dis. 20:1531-1534 and Dean, D.A., Burckard, K. W. 1996, “Fungal infection in surgical patients” Am. J. Surg. 171: 374-382.

Aspergillosis is an infectious disease which is caused by the genus Aspergillus and leads mainly to disorders of the respiratory organs, but also of the skin and other organs.

Against this background, efforts were made at an early date to develop reliable methods for detecting fungi in clinical material. In this connection, during the last 10 years cultivation and histopathological methods have increasingly been replaced, because of their limited sensitivity and specificity, by molecular biological detection methods. Of these methods, the optimal diagnostic approach appears to be a polymerase chain reaction (PCR)-assisted detection of fungal nucleic acids since (i) it is considerably more sensitive than current cell culture-based methods, (ii) it makes it possible to detect many different fungal genera, (iii) it can be applied to a large number of different types of samples, and (iv) it provides reliable results within a short time, so that a goal-oriented therapeutic strategy can be initiated at an early time.

From DE 195 30 332 C2, DE 195 30 33 C2 and DE 195 30 336 C2 various nucleic acids are known with which a PCR-assisted detection of fungal DNA of various fungal species is possible. These publications describe two nucleic acid molecules which have the sequences SEQ ID No. 5 and 6 from the sequence listing appended hereto, which can be employed as PCR primer pair and demonstrably detect fungal DNA of the widespread human-pathogenic fungal species Candida albicans, Candida glabrata, Candida krusei, Candida tropicalis, Candida parapsilosis, Aspergillus fumigatus, Aspergillus niger, Aspergillus flavus, Aspergillus terreus, and Aspergillus nidulans. These publications describe further six nucleic acid molecules which can be used as hybridization probes for specific detection of Candida albicans, Candida glabrata, Candida krusei, Candida tropicalis, Candida parapsilosis and of the genus Aspergillus.

DE 196 35 347 C1 discloses the nucleotide sequences of four further hybridization probes with which species-specific detection of the fungal DNA from Pneumocystis karnii, Malassezia furfur (SEQ ID No. 18 from the sequence listing appended hereto), Trichosporum cuaneum/Trichosporum capitatum (SEQ ID No. 19 from the sequence listing appended hereto) and of Fusarium solani/Fusarium oxysporum succeeds.

Einsele et al., 1997, “Detection and identification of fungal pathogens in blood by using molecular probes”, J. Clin. Microbiol. 35:1353-1360, developed a PCR primer pair which binds to the 18S rRNA gene which is highly conserved in the fungal kingdom. The authors are able by using this primer pair within the scope of a detection method for the presence of fungal DNA to detect the following 22 different fungal species in patients' blood samples, especially of very widespread Candida and Aspergillus species: Candida albicans, Candida tropicalis, Candida parapsilosis, Candida glabrata, Candida krusei, Candida guillermondii, Candida kefyr, Aspergillus fumigatus, Aspergillus flavus, Aspergillus terreus, Aspergillus niger, Aspergillus nidulans, Aspergillus versicolor.

Van Burik et al., 1998 “Panfungal PCR assay for detection of fungal infections in human blood specimens” J. Clin. Microbiol. 36:1196-1175 likewise describes a PCR primer pair which binds to the 18S rRNA gene and by means of which it is possible to detect 40 different fungal species in patients' whole blood. These likewise include in particular widespread Candida, Aspergillus and also Fusarium and Microsporum species.

A crucial disadvantage of these known nucleic acid molecules and methods respectively, is, however, that it is not possible therewith to detect fungal species which were for a long time insignificant but are now becoming increasingly important, e.g., rare Candida species. The result of this is that a false-negative diagnosis of a fungal infection occurs in the event that such a species is present. In order to be able also to detect such previously unusual fungal species, in the prior art, the PCR-assisted methods were carried out in parallel to cultivations, or serological detections which again have the abovementioned disadvantages such as lack of sensitivity and specificity, false-negative determinations, high consumption of time, etc.

### SUMMARY OF THE INVENTION

It is therefore an object of the present invention to provide a nucleic acid molecule of the type mentioned at the outset, with which the abovementioned disadvantages are avoided.

This object is achieved with the nucleic acid molecule that has one of the following nucleotide sequences: SEQ ID No. 1 to SEQ ID No. 4 or SEQ ID No. 7 to SEQ ID No. 17 from the appended sequence listing or a sequence which binds to such a sequence to which also one of the sequences SEQ ID No. 1 to SEQ ID No. 4 or SEQ ID No. 7 to SEQ ID No. 17 binds.

This object on which the invention is based is completely achieved thereby.

This is because the inventors have realized that, using the aforementioned nucleotide sequences, on the one hand fungal DNA from the entire range of fungi is detected, and on the other hand species-specific detection of fungal DNA is possible in particular of species which have previously been relatively insignificant but are becoming increasingly important, such as, for example, certain Candida species.

The underlying object is achieved according to the invention not only by a nucleic acid molecule having one of the sequences SEQ ID Nos. 1 to 4 or SEQ ID No. 7 to SEQ ID No. 17 from the appended sequence listing, but also by such a nucleic acid molecule which binds to the same sequences to which also the nucleic acid molecule having one of the sequences SEQ ID Nos. 1 to 4 or SEQ ID No. 7 to SEQ ID No. 17 binds.

This encompasses in particular those nucleic acid molecules where the sequences listed in the sequence listing are a constituent of a longer sequence. Even if a few nucleotide exchanges have been carried out on the sequences listed in the sequence listing, these molecules generally retain their highly selective affinity for the fungal DNA. Consequently, a further object of the present invention is a nucleic acid molecule characterized in this way.

The preceding statements apply to all nucleic acid molecules of the invention and nucleotide sequences from the appended sequence listing, respectively, and to their use in the methods according to the invention.

A nucleic acid molecule having one of the sequences SEQ ID Nos. 1 to 10 is particularly suitable for detecting a general fungal infection. This is because this nucleic acid molecule binds to the 18S rRNA gene which is highly conserved in the entire fungal kingdom. A database analysis in the Blast Search program of the National Center for Biotechnology Information showed homologies to more than 500 different fungal species via comparative sequence analysis of the sequences SEQ ID Nos. 1 to 10. The inventors conclude therefrom that detection of fungal DNA from the entire fungal kingdom is possible with the aid of the aforementioned nucleic acid molecule.

This ensures detection even of fungal species which have previously been relatively insignificant or unusual. Because the binding properties of the aforementioned nucleic acid molecule are present even under stringent conditions, the latter can be used for example as polymerase chain reaction (PCR) primer for the amplification of amounts of fungal DNA present in low concentration or for the sequencing of previously unknown fungal 18S rRNA genes.

A nucleic acid molecule having one of the nucleotide sequences SEQ ID Nos. 1 to 10 is thus very particularly suitable for rapid and reliable assessment of a possible general fungal infection on the basis of clinical material, for example blood or tissue samples, irrespective of the causative fungal species.

A nucleic acid molecule having one of the nucleotide sequences SEQ ID No. 11 to SEQ ID No. 19 from the appended sequence listing is, by contrast, particularly suitable, for example, for use as hybridization probe, e.g., within the scope of a species-specific analysis for the presence of rare Candida species.

On the basis of the above statements, another object of the present invention is a method for detecting fungi in clinical material with the steps: a) provision of fungal DNA from clinical material, b) detection of the fungal DNA, wherein the detection in step b) takes place by using the nucleic acid molecule according to the invention.

It is a further object of the invention to detect fungi via amplification of a fungal DNA segment by means of a polymerase chain reaction (PCR) in which the primers of a PCR primer pair have nucleotide sequences in a combination which is selected from the group consisting of: SEQ ID Nos. 1 and 2; SEQ ID Nos. 3 and 4; SEQ ID Nos. 7 and 8; SEQ ID Nos. 9 and 10 from the appended sequence listing; two sequences which bind to sequences to which also SEQ ID Nos. 1 and 2 or SEQ ID 3 and 4 or SEQ ID Nos. 7 and 8 or SEQ ID Nos. 9 and 10 bind.

This has the particular advantage that segments of fungal DNA, which are frequently present only in small amounts in clinical material, are enriched so greatly that they can be detected by simple methods, e.g., ethidium bromide staining after agarose gel electrophoresis.

Nucleic acid molecules having the nucleotide sequences SEQ ID Nos. 1 to 10 listed in the sequence listing have been successfully tested by the inventors as PCR primers, i.e. their use for detecting fungi provides reliable results in a highly specific and sensitive manner for each of these nucleic acid molecules. When they are used in a PCR there is neither coamplification of human nucleic acids nor of bacterial or viral nucleic acids which are possibly present.

It is expedient in this connection according to the teaching of the invention to use as PCR primer pair nucleic acid molecules having the nucleotide sequences as in the stated combination. Particularly suitable for amplification of the fungal DNA segment are alternatively five different PCR primer pairs:

PCR primer pair No. 1: SEQ ID No. 1 and SEQ ID No. 2;

PCR primer pair No. 2: SEQ ID No. 3 and SEQ ID No. 4;

PCR primer pair No. 3: SEQ ID No. 5 and SEQ ID No. 6;

PCR primer pair No. 4: SEQ ID No. 7 and SEQ ID No. 8; or

PCR primer pair No. 5: SEQ ID No. 9 and SEQ ID No. 10.

It is, of course, also possible here to use as PCR primer pair nucleic acid molecules in appropriate combination with nucleotide sequences which bind to nucleotide sequences to which one of the nucleotide sequences SEQ ID Nos. 1 to 10 binds. The nucleic acid molecules meant in this connection are, in particular, those with which one of the sequences listed in the sequence listing is a constituent of a longer sequence, or with which in isolated cases point mutations and/or deletions, substitutions, additions of nucleotides etc., have taken place compared with the sequences indicated in the sequence listing. The reason for this is that these modifications generally have no influence on the function according to the invention of the molecules and sequences, respectively, listed in the sequence listing, so that the present application also relates to the use of such molecules.

The use of nucleic acid molecules as PCR primers in the combination indicated above leads to optimal amplicon sizes and thus ensures reliable detection of fungal DNA present. Accordingly, a further subject matter of the present invention is a use in which the nucleic acid molecules having the sequences of the invention are employed as primer pairs in the combination mentioned.

It is another object of the invention to carry out within the method according to the invention a species-/or genus-specific analysis of the fungal DNA by hybridization with a nucleic acid molecule which has one of the nucleotide sequences which is selected from the group consisting of: SEQ ID No. 11 to SEQ ID No. 17 from the appended sequence listing and a sequence which binds to such a sequence to which also one of the sequences SEQ ID No. 11 to SEQ ID NO. 17 binds.

This has the particular advantage that, for example, after a positive finding of a general fungal infection the analysis necessary for a specific therapy, or the detection of the causative fungal species, is made possible. This is attributable to the fact that a nucleic acid molecule having any of the aforementioned nucleotide sequences shows a specific affinity for a defined fungal species DNA even under stringent conditions. This makes the aforementioned nucleic acid molecule particularly suitable as hybridization probe.

It has proved particularly beneficial in this connection to label the aforementioned nucleic acid molecule or the hybridization probe at its 5′ end with digoxigenin and to use it in a slot-blot assay. In this assay, the fungal DNA is transferred with the aid of a slot plate as mask onto a nylon support membrane, and the membrane is then incubated with the appropriately labeled hybridization probes. After subsequent washing steps under stringent conditions, hybridization to the fungal DNA can be identified by means of an optically detectable signal, with visual quantification being facilitated by the sample concentration on a small area. Use of the aforementioned nucleic acid molecule in such a slot-blot assay allows a particularly high sensitivity of the detection method to be reached, i.e. as far as a limit of detection of 100 fg of fungal DNA in clinical material.

It is another object of the invention to carry out within the method according to the invention a detection of Candida DNA by using a nucleic acid molecule which has the nucleotide sequence SEQ ID No. 11 from the appended sequence listing or a sequence which binds to such a sequence to which also the sequence SEQ ID No. 11 binds.

The inventors have in this way succeeded for the first time in developing a hybridization probe with which it is possible to detect a genus-specific Candida DNA, irrespective of the particular Candida species.

In the prior art, a general candidal infection, for example an invasive candidiasis, is usually diagnosed clinically via methods which include the cultivation of fungal cells. A particular problem in this connection is that a result of such a cultivation mixture can be achieved only after several days. It is often too late for effective treatment of a candidal infection after this time. A further problem is that Candida often, as a commensal of the human skin, contaminates the cultivation mixtures and gives false-positive results. Even more of a problem is the fact that in up to 50 percent of cases of systemic candidiasis checked by autopsy the underlying blood cultures were negative, which means that this method has no diagnostic value at all. This has led to the widespread opinion among clinicians that invasive candidal infections can always be diagnosed only by autopsies.

The use of the so-called genus-specific Candida hybridization probe described above provides an effective remedy for this. It is also suitable, in contrast to NMR and radioisotope scanning methods frequently performed at present, for detecting a candidal infection even at an early stage.

A method based on the Candida metabolite arabinitol as diagnostic marker had to be abandoned as unsuitable after it was discovered that arabinitol is likewise produced by the human body.

In this respect too, the aforementioned object of the invention provides a remedy, because absolutely no cross-reaction of the genus-specific Candida hybridization probe with human nucleic acid is observed. Even the use of the two nucleic acid molecules described in DE 195 30 332 C2 as PCR primers does not make it possible to detect a general Candida infection, because DNA of the genera Aspergillus is also amplified by these primers. In addition, it has been demonstrated that only five different Candida species are detected thereby. Thus, the genus-specific Candida hybridization probe of the invention makes it possible for the first time to detect a general candidal infection reliably and at an early time.

In connection with further analysis, i.e. the species-specific determination of the fungal DNA present in various preferred embodiments as a function of the species to be detected, it is another object of the invention to use within the method according to the invention a nucleic acid molecule as hybridization probe having the following nucleotide sequence:

nucleotide sequence SEQ ID No. 12 for detecting Candida humicola DNA,

nucleotide sequence SEQ ID No. 13 for detecting Candida inconspicua DNA,

nucleotide sequence SEQ ID No. 14 for detecting Candida lusitaniae DNA,

nucleotide sequence SEQ ID No. 15 for detecting Candida norwegensis and/or Candida krusei DNA,

nucleotide sequence SEQ ID No. 16 for detecting Candida kefyr DNA, nucleotide sequence SEQ ID No. 17 for detecting Candida solani DNA.

For the reasons stated at the outset, a further subject matter of the present invention in this connection is the corresponding use of a nucleic acid molecule within the method according to the invention, for detecting one of the aforementioned species, wherein said molecule has a sequence which binds to such a sequence to which also one of the sequences SEQ ID Nos. 11 to 17 from the sequence listing binds.

A further object of the present invention is a method for determining the sequence of ribosomal fungal genes, with the steps: a) provision of fungal DNA from clinical material; b) detection of the fungal DNA, and c) sequencing the fungal DNA detected in step b), wherein the detection in step b) takes place by using at least one nucleic acid molecule which is selected from the group consisting of: SEQ ID No. 1 to SEQ ID No. 4, SEQ ID No. 7 to SEQ ID No. 10, and a sequence which binds to such a sequence to which also one of the foregoing sequences binds.

As mentioned at the outset, the stated nucleic acid molecules can be used for example as PCR primers in a combination which is likewise stated for elucidating the nucleotide sequence of previously unknown ribosomal fungal genes. This is equally attributable to the ability of the stated molecules to bind to highly conserved sections of all 18S rRNA fungal genes.

It is possible on the basis of the explained favorable properties of the claimed nucleic acid molecule to detect in the methods according to the invention in a reliable and time-saving way fungi, which are present in a large number of different clinical materials, e.g. whole blood or tissue samples, for example, as a result of a fungal infection, by means of their DNA. In contrast to the previous methods employed clinically and described at the outset, a PCR-assisted detection of a general fungal infection succeeds for the first time, whereby fungal species can be generally detected, which have been relatively rare to date from the entire fungal kingdom, and whereby a further species-specific detection of, for example, rare Candida species being enabled.

The methods of the invention particularly aim at providing not only DNA from fungal cells which are present free in the blood but also from those fungal cells which have been phagocytosed by certain blood cells, e.g. granulocytes or monocytes, but also macrophages in the tissue. It is therefore possible by such methods to diagnose fungal infections early, i.e. even in a stage at which only a few or no fungal cells at all are present free in the blood because of the early immune response.

Another object of the invention relates to a kit which comprises a nucleic acid molecule which has one of the following nucleotide sequences selected from the group consisting of: SEQ ID No. 1 to SEQ ID No. 4 and SEQ ID No. 7 to SEQ ID No. 17 from the appended sequence listing and a sequence which binds to such a sequence to which also one of the sequences SEQ ID No. 1 to SEQ ID No. 4 or SEQ ID No. 7 to SEQ ID NO. 17 binds.

Another object of the present invention relates to a kit for carrying out the methods according to the invention. The provision of such kits have the advantage that possible errors in carrying out the method or the use according to the invention of the claimed nucleic acid molecule are avoided through the assembling of all the reagents and/or nucleic acid molecules, reaction containers or parts thereof, of detailed instructions for use, etc. This takes account in particular of the situation in clinics where frequently semiskilled staff are entrusted with carrying out such methods.

It will be appreciated that the abovementioned features can be used not only in the stated combination but also singly or in another combination without departing from the scope of the present invention.

### BRIEF DESCRIPTION OF THE DRAWINGS

FIG. 1 shows a slot-blot assay using the general Candida probe having the nucleotide sequence SEQ ID No. 11.

FIG. 2 shows a slot-blot assay using 6 different species-specific probes having the nucleotide sequences SEQ ID Nos. 12 to 17.

### DETAILED DESCRIPTION OF THE PREFERRED EMBODIMENT

### EXAMPLE 1

Obtaining of Fungal DNA

A blood sample is taken from patients to be investigated for a fungal infection. The erythrocytes from the whole blood are lysed hypotonically with RCLB buffer (10 mM Tris [pH 7.6], 5 mM MgCl₂, 10 mM NaCl). This is followed by enzymatic lysis of the leukocytes with WCLB buffer (10 mM Tris[pH 7.6], 10 mM EDTA, 50 mM NaCl, 0.2% SDS, 200 μg of proteinase K per ml) at 65° C. for 45 minutes. This ensures detection also of fungal DNA derived from fungal cells located inside blood cells. The samples are pelleted and incubated with 50 mM NaOH at 95° C. for 10 minutes. This is followed by neutralization with 1 M Tris [pH 7.0], followed by treatment with recombinant lyticase (Sigma, Deissenhofen, Germany) in a buffer which contains 1 U of lyticase per 100 μl, 50 mM Tris [pH 7.5], 1 mM EDTA and 0.2% β-mercaptoethanol at 37° C. for 45 minutes in order to form spheroplasts. After centrifugation at 5000 g, the supernatant, which contains the human DNA and proteins, is decanted off and the pellets are treated with 1 M Tris-EDTA and 10% SDS at 65° C. for 30 minutes to lyse the spheroplasts. 5 M potassium acetate is then added, and the samples are incubated at −20° C. for 30 minutes to precipitate proteins. After a further centrifugation step at 100 g for 20 minutes, the DNA is precipitated from the supernatant with cold isopropanol. The DNA is purified with 70% strength ethanol, dried in air and resuspended in 40 μL of H₂O. After spectrophotometry, the samples are diluted to a final concentration of 50 ng of DNA per μl.

### EXAMPLE 2

R for Detecting the Fungal DNA

The samples obtained in Example 1 are examined for the presence of fungal DNA by means of a polymerase chain reaction (PCR} in which there is specific exclusive amplification of fungal DNA. Suitable PCR primers are shown in Table I.

(i) PCR Primer Sequences of the Five Primer Pairs.

TABLE I

PCR primer sequences for detecting fungal DNA

| | | | | Hybridi- |
|---|---|---|---|---|
| Primer | | | | zation |
| pairs | | Tm | GC | temperature |
| SEQ ID | Primer sequences | [°C.] | [%] | [°C.] |

| | | | | |
|---|---|---|---|---|
| 1 | 5′-GATCCTGCCAGTAGTCATATG | 62 | 48 | 55 |
| 2 | 5′-CTATCCTACCATCGAAAGTTG | 60 | 43 | 55 |
| 3 | 5′-GGTTCATTCAAATTTCTGCC | 56 | 40 | 54 |
| 4 | 5′-CACCAGACTTGCCCTCC | 56 | 65 | 54 |
| 5 | 5′-ATTGGAGGGCAAGTCTGGTG | 62 | 55 | 62 |
| 6 | 5′-CCGATCCCTAGTCGGCATAG | 64 | 60 | 62 |
| 7 | 5′-TAGGGGATCGAAGATGATCA | 59 | 45 | 56 |
| 8 | 5′-GACCTGGTGAGTTTCCCCG | 62 | 58 | 56 |
| 9 | 5′-ATTGACGGAAGGGCACCAC | 60 | 58 | 60 |
| 10 | 5′-TGTACAAAGGGCAGGGACG | 60 | 58 | 60 |

Tₘ-melting temperature
GC = guanine/cytosine content of the sequence

The amplification reactions are carried out in a volume of 50 μl which contains: 10 mM Tris [pH 9.6], 50 mM NaC1, 10 mM MgCl₂, 200 μg of bovine serum albumin/ml, 0.5 mM deoxyribonucleotide trisphosphates, 100 pmol of the respective primers and 1.5 U of Taq polymerase (Amersham, Braunschweig, Germany). The extracted DNA from Example 1 (100 ng) is added and 34 cycles of repeated denaturation, primer hybridization enzymatic chain extension are carried out in a PE 2400 Thermocycler (Perkin Elmer, Dreieich, Germany). The amplification program in this case has the following profile: 30 seconds at 94° C., 1 minute at 62° C. and 2 minutes at 72° C., followed by a cycle of terminal extension at 72° C. for 5 minutes. In order to detect possible contamination, aliquot of a sodium chloride solution and of human fibroblast DNA are prepared and employed in the same way as negative control in the amplification reaction.

(iii) Detection of Amplified Fungal DNA

Fungal DNA is detected from fractionating 10 μl aliquots of each amplification product by electrophoresis on a 2% agarose gel in 1×TAE buffer (pH 8.0, 40 mM Tris-acetate [pH 7.5], 2 mM sodium EDTA), followed by ethidium bromide staining. Excitation of the ethidium bromide intercalated into the DNA allows the fluorescent fungal DNA to be demonstrated on a screen.

The five indicated PCR primer pairs are thus suitable for detecting in a simple manner the presence of fungi or fungal DNA, for example in patients' blood, irrespective of the particular fungal species.

### EXAMPLE 3

Analysis of Fungal DNA

A slot-blot assay is carried out for further determination of the species and genus of fungus. For this purpose, 10 μl aliquots of each amplicon from Example 2 are pipetted onto Hybond N+ nylon membranes (Amersham, Braunschweig, Germany) on which a slot plate is placed.

The table below shows the sequences of the digoxigenin-labeled hybridization probes, the corresponding melting temperatures at which half of the respective nucleic acid molecule in a solution is in the double-stranded form and the other half is in the form of single-stranded molecule, the respective GC content, i.e. the quantity of guanine and cytosine residues in the molecule, and the specific washing temperature (see hereinafter).

TABLE II

Probe sequences for the species/genus-specific
analysis of fungal DNA

| Probes | | | | Washing |
|---|---|---|---|---|
| SEQ | | Tₘ | GC | temperature |
| ID No. | Probe sequences | [°C.] | [%] | [°C.] |
| 12 | 5′-Dig-CGTATGCCCTTCATTGGGTGTGC | 72 | 57 | 70 |
| 13 | 5′-Dig-TACCTATGGTGAGTACTGCTGC | 66 | 46 | 63 |
| 14 | 5′-Dig-CGTCCGCTTAGGCGAGCACTG | 70 | 67 | 68 |
| 15 | 5′-Dig-TACCTATGGTAAGCACTGTTGC | 64 | 46 | 62 |
| 16 | 5′-Dig-ACCTGTACTCCTTGTGGGTGCA | 68 | 56 | 65 |
| 17 | 5′-Dig-CGCTTTTTTGCGAGTACTGGAC | 66 | 50 | 63 |
| 18 | 5′-Dig-AGAGTGTTCAAAGCAGGCTT(K)ACGCC | 74 | 54 | 72 |
| 19 | 5′-Dig-AGGCCGTATGCCCTTCATTGGGTGTGCGGT | 78 | 60 | 75 |
| 11 | 5′-Dig-GGACCATCGTAATGATTAATAGGGACG | 78 | 44 | 73 |

Tₘ = melting temperature
GC = guanine/cytosine content of the sequence
Dig = digoxigenin 5-phosphate

Each amplicon (Example 2) is hybridized with the respective probe at 42° C. for 20 minutes. This is followed by specific washing steps with washing buffer (100 mM sodium chloride, 10 mM sodium dihydrogen phosphate, 1 mM EDTA, 1% SDS) twice at the above mentioned probe-specific temperatures for 7 minutes. The washing temperature which is only a few degrees below the Tm, and the presence of SDS in the washing buffer, in this case permit only specific hybridization reactions and prevent nonspecific annealing of probes. The hybrids are then incubated subsequently with anti-digoxigenin antibodies conjugated with alkali phosphatase (Roche, Molecular Biochemicals, Mannheim, Germany) for 20 minutes, and for a further 30 minutes with nitroblue tetrazolium (75 mg/ml in dimethylformamide) and promochlor-indoyl phosphate solution (50 mg/ml in dimethylformamide, Roche Molecular Biochemicals, Mannheim, Germany).

In the event of hybridization of the probe, the by subsequent enzymatic cleavage of a chromogenic substrate by the enzyme bound to the antibody forms a colored reaction product which indicates a positive reaction for the particular mixture or for the particular amplicon. If no hybridization has taken place because either there has been amplification of fungal DNA from a different species than one for which the probe is specific, or no fungal DNA was present in the clinical material, consequently no anti-digoxigenin antibody can bind to the labeled probe and is washed away together with the latter. The colored reaction product is not formed, and thus the reaction is assessed as negative.

### EXAMPLE 4

Specificity of the Hybridization Probes

To check the specificity of the claimed hybridization probes, the following yeast cultures were obtained from the Deutsche Sammlung von Mikroorganismen (DMSZ, Braunschweig, Germany):

Candida albicans (DSM 6569), Candida glabrata (DSM 6425), Candida krusei (DSM 6128), Candida tropicalis (DSM 5991), Candida parapsilosis (DSM 5784), Candida lusitanie (DSM 70102), Candida humicola (DSM 5572), Candida pseudotropicalis (ΔCandida kefyr) (ATTC 14438), Candida incosnspicua (DSM 70631), Candida solani (DSM 3315), Candida norvegensis (DSM 70760), Candida utilis (DSM 2361), Saccharomyces cerisiae (DSM 1333), Trichosporon cutaneum (DSM 70698), Malassezia furfur (DSM 6170), Fusariumsol solani (DSM 1164) and Aspergillus fumigatus (DSM 790). The yeast cells were washed and resuspended in 0.9% strength sterile sodium chloride solution. The yeast cell suspensions were titrated to adjust to final concentrations of 10⁶ to 10¹ colony-forming units (CFU) per ml of solution. In addition, 100 μl of the suspension containing 10⁵ to 10¹ CFU were added to blood samples obtained from healthy volunteers. Ten further samples from colonized patients (n=5) and patients with systemic fungal infections (n=4) were obtained from the Huddinge Hospital, Huddinge, Sweden, and from the Hygiene Institute, Tubingen University, Germany. In addition, yeasts were isolated from feces, liver, abscesses, sputum or blood from patients suffering from lymphoma, acute lymphatic leukemia or AIDS.

It emerges that the genus-specific Candida probe (with the sequence SEQ ID No. 11) surprisingly detected DNA from all 15 analyzed yeast species (C. albicans, C. glabrata, C. krusei, C. tropicalis, C. parapsilosis, C. lusitaniae, C. humicola, C. pseudotropicalis (ΔC. kefyr), C. inconspicua, C. solani, C. norwegensis, C. utilis, S. cerevisiae, T. cutaneum and M. furfur). No hybridization signal was obtained for DNA isolated from Aspergillus fumigatus, Aspergillus niger, Fusarium ssp., cytomegalovirus and from human fibroblasts.

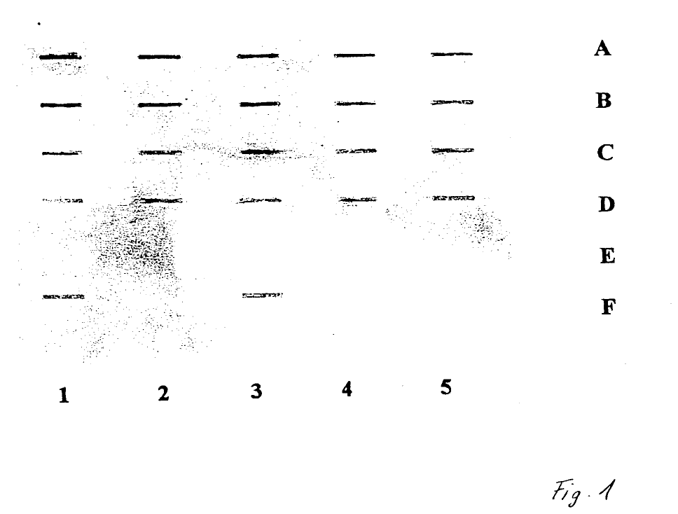

The result of such experiment or slot-blot assay is depicted in FIG. 1. The following quantities of C. lusitaniae DNA were loaded in lane 1: 100 pg (A), 10 pg (B), 1 pg (C), 100 fg (D); corresponding quantities of C. tropicalis DNA were loaded in lane 2; corresponding quantities of C. glabrata DNA in lane 3; corresponding quantities of C. krusei DNA in lane 4; corresponding quantities of C. parapsilosis DNA in lane 5. Aliquots of clinical isolates infected with corresponding fungal cultures were loaded at points F: C. lusitaniae isolate (F1), C. glabrata isolate (F3), A. fumigatus isolate (F4, negative control), A. niger isolate (F5, negative control); double-distilled water was additionally loaded as further negative control (F2).

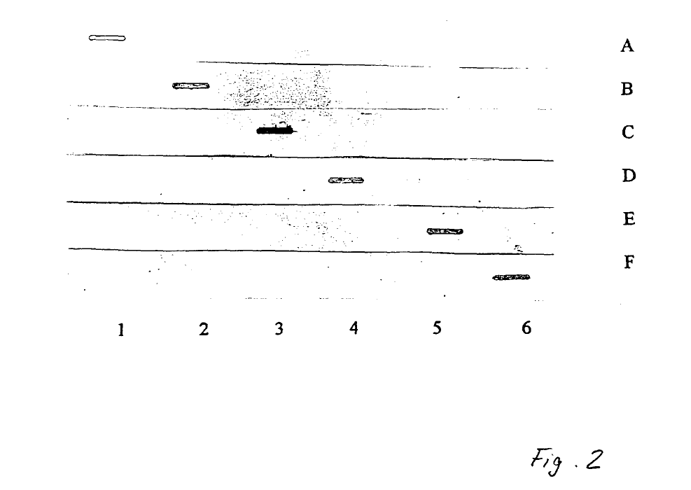

The species-specific hybridization probes were highly specific even under the stringent washing conditions described above. In addition, no cross-reactions at all were observed within the abovementioned species with the hybridization probes comprising one of the sequences SEQ ID Nos. 12 to 14, 16 to 19; with the exception of the hybridization probe comprising the sequence SEQ ID No. 15, which recognizes both DNA from C. norvegensis and from C. krusei. The latter is depicted in FIG. 2. In this case, 10 pg of DNA from each of the following fungal species were loaded: C. humicola (1), C. solani (2), C. inconspicua (3), C. norvegensis (4), C. kefyr (5) and C. lusitaniae (6). The corresponding membrane strips were incubated with the following species-specific probes: SEQ ID No. 12 (A), SEQ ID No. 17 (B), SEQ ID No. 13 (C), SEQ ID No. 15 (D), SEQ ID No. 16 (E) and SEQ ID No. 14 (F).

All ten isolates from the nine patients with superficial or invasive infection by C. lusitaniae (n=2), C. inconspicua (n=1), C. norwegensis (n=1), C. glabrata (n=3), C. albicans (n=3) gave positive signals both with the genus-specific Candida probe (with the sequence SEQ ID NO. 11; see also FIG. 1, F1, F3) and with the corresponding species-specific hybridization probes (with the sequences SEQ ID No. 12 to SEQ ID No. 19).

### EXAMPLE 5

Sensitivity of the Hybridization Probes

To determine the sensitivity of the assay, a titration was carried out with various Candida species cultures (C. albicans, C. humicola, C. lusitaniae, C. inconspicua, C. norwegensis, C. pseudotropicalis (ΔC. kefyr), C. solani), the concentrations being adjusted to 10⁵ to 10⁰ CFU. This showed a lower limit of detection of at least 10¹ CFU, which corresponds to an absolute quantity of 100 fg of fungal DNA. The inventors were able to document this high sensitivity for all the species-specific probes (with the sequences SEQ ID No. 12 to SEQ ID No. 19) and for the genus-specific Candida probe (with the sequence SEQ ID No. 11; see also FIG. 1).

What is claimed is:

1. A nucleic acid molecule, which comprises a nucleotide sequence selected from the group consisting of: SEQ ID No. 1, SEQ ID No. 2, SEQ ID No. 3, SEQ ID No. 4, SEQ ID No. 7, SEQ ID No. 8, SEQ ID No. 9, SEQ ID No. 10, SEQ ID No. 11, SEQ ID No. 12, SEQ ID No. 13, SEQ ID No. 14, SEQ ID No. 15, SEQ ID No. 16, SEQ ID No. 17, and a sequence which binds to such a sequence to which one of the sequences SEQ IDs No. 1-4 or SEQ ID Nos. 7-17 binds.

2. A method for detecting a fungus in a clinical material, comprising:
a) obtaining a fungal DNA from said clinical material,
b) detecting the fungal DNA, wherein said detecting is performed using a nucleic acid molecule, which comprises a nucleotide sequence selected from the group consisting of: SEQ ID No. 1, SEQ ID No. 2, SEQ ID No. 3, SEQ ID No. 4, SEQ ID No. 7, SEQ ID No. 8, SEQ ID No. 9, SEQ ID No. 10, SEQ ID No. 11, SEQ ID No. 12, SEQ ID No. 13, SEQ ID No. 14, SEQ ID No. 15, SEQ ID No. 16, SEQ ID No. 17, and a sequence which binds to such a sequence to which one of the sequences SEQ IDs No. 1-4 or SEQ ID Nos. 7-17 binds.

3. The method of claim 2, wherein the detecting of the fungal DNA is performed by amplification of a segment of the fungal DNA by polymerase chain reaction (PCR) using a primer pair selected from the group consisting of: SEQ ID Nos. 1 and 2; SEQ ID Nos. 3 and 4; SEQ ID Nos. 7 and 8; SEQ ID NO. 9 and 10; and two sequences which bind to such sequences to which SEQ ID Nos. 1 and 2, or SEQ ID NO. 3 and 4, or SEQ ID Nos. 7 and 8, or SEQ ID Nos. 9 and 10 bind, thereby obtaining a PCR amplicon.

4. The method of claim 2, wherein detecting of a Candida DNA is performed using a nucleic acid molecule, which comprises the nucleotide sequence of SEQ ID No. 11.

5. The method of claim 2, wherein detecting of a Candida DNA is performed using nucleic acid molecule which comprises a sequence which binds to a sequence to which the sequence of SEQ ID No. 11 binds.

6. The method of claim 2, wherein detecting of a Candida humicola DNA is performed using a nucleic acid molecule which comprises the nucleotide sequence of SEQ ID No. 12.

7. The method of claim 2, wherein detecting of a Candida inconspicua DNA is performed using a nucleic acid molecule which comprises the nucleotide sequence of SEQ ID No. 13.

8. The method of claim 2, wherein detecting of a Candida lusitaniae DNA is performed using a nucleic acid molecule which comprises the nucleotide sequence of SEQ ID No. 14.

9. The method of claim 2, wherein detecting of a Candida norwegensis DNA and/or Candida krusei DNA is performed using a nucleic acid molecule which comprises the nucleotide sequence of SEQ ID No. 15.

10. The method of claim 2, wherein detecting of a Candida kefyr DNA is performed using a nucleic acid molecule which comprises the nucleotide sequence of SEQ ID No. 16.

11. The method of claim 2, wherein detecting of a Candida solani DNA is performed using a nucleic acid molecule which comprises the nucleotide sequence of SEQ ID No. 17.

12. The method of claim 3, further comprising performing a species-, a genus-, or a combination thereof-specific analysis of the fungal DNA by hybridization of said PCR amplicon to a nucleic acid molecule which comprises a nucleotide sequence selected from the group consisting of: SEQ ID No. 11, SEQ ID No. 12, SEQ ID No. 13, SEQ ID No. 14, SEQ ID No. 15, SEQ ID No. 16, SEQ ID No. 17, and a sequence which binds to a sequence to which one of the sequences SEQ ID No. 11 to SEQ ID No. 17 binds.

13. The method of claim 3, further comprising performing a species-, a genus-, or a combination thereof-specific analysis of the fungal DNA by hybridization of said PCR amplicon with a nucleic acid molecule which comprises a nucleotide sequence selected from the group consisting of: SEQ ID No. 11, SEQ ID No. 12, SEQ ID No. 13, SEQ ID No. 14, SEQ ID No. 15, SEQ ID No. 16, SEQ ID No. 17, and a sequence which binds to a sequence to which one of the sequences SEQ ID No. 11 to SEQ ID No. 17 binds.

14. The method of claim 3, wherein detecting of a Candida DNA is performed using a nucleic acid molecule, which comprises the nucleotide sequence of SEQ ID No. 11.

15. The method of claim 3, wherein detecting of a Candida DNA is performed using nucleic acid molecule which comprises a sequence which binds to a sequence to which the sequence of SEQ ID No. 11 binds.

16. The method of claim 3, wherein detecting of a Candida humicola DNA is performed using a nucleic acid molecule which comprises the nucleotide sequence of SEQ ID No. 12.

17. The method of claim 3, wherein detecting of a Candida inconspicua DNA is performed using a nucleic acid molecule which comprises the nucleotide sequence of SEQ ID No. 13.

18. The method of claim 3, wherein detecting of a Candida lusitaniae DNA is performed using a nucleic acid molecule which comprises the nucleotide sequence of SEQ ID No. 14.

19. The method of claim 3, wherein detecting of a Candida norwegensis DNA and/or Candida krusei DNA is performed using a nucleic acid molecule which comprises the nucleotide sequence of SEQ ID No. 15.

20. The method of claim 3, wherein detecting of a Candida kefyr DNA is performed using a nucleic acid molecule which comprises the nucleotide sequence of SEQ ID No. 16.

21. The method of claim 3, wherein detecting of a Candida solani DNA is performed using a nucleic acid molecule which comprises the nucleotide sequence of SEQ ID No. 17.

22. A method for determining a sequence of a ribosomal fungal gene, comprising:
a) obtaining a fungal DNA from a clinical material;
b) detecting the fungal DNA, and
c) sequencing the fungal DNA detected in step b),
wherein said detecting is performed by using at least one nucleic acid molecule, which comprises a nucleotide sequence selected from the group consisting of: SEQ ID No. 1, SEQ ID No. 2, SEQ ID No. 3, SEQ ID No. 4, SEQ ID No. 7, SEQ ID No. 8, SEQ ID No. 9, SEQ ID No. 10, SEQ ID No. 11, SEQ ID No. 12, SEQ ID No. 13, SEQ ID No. 14, SEQ ID No. 15, SEQ ID No. 16, SEQ ID No. 17, and a sequence which binds to such a sequence to which one of the sequences SEQ IDs No. 1-4 or SEQ ID Nos. 7-17 binds.

23. A method for determining a sequence of a ribosomal fungal gene, comprising:
a) obtaining a fungal DNA from a clinical material;
b) detecting the fungal DNA, and
c) sequencing the fungal DNA detected in step b),
wherein said detecting is performed by amplification of a segment of the fungal DNA by a polymerase chain reaction {PCR) in which the PCR primer pair is selected from the group consisting of: SEQ ID Nos. 1 and 2; SEQ ID Nos. 3 and 4;
SEQ ID NOS. 7 and 8; SEQ ID Nos. 9 and 10; and two sequences which bind to such sequences to which SEQ ID Nos. 1 and 2, or SEQ ID Nos. 3 and 4, or SEQ ID Nos. 7 and 8, or SEQ ID Nos. 9 and 10 bind.

24. A kit, comprising a nucleic acid molecule, which comprises a nucleotide sequence selected from the group consisting of: SEQ ID No. 1, SEQ ID No. 2, SEQ ID No. 3, SEQ ID No. 4, SEQ ID No. 7, SEQ ID No. 8, SEQ ID No. 9, SEQ ID No. 10, SEQ ID No. 11, SEQ ID No. 12, SEQ ID No. 13, SEQ ID No. 14, SEQ ID No. 15, SEQ ID No. 16, SEQ ID No. 17, and a sequence which binds to such a sequence to which one of the sequences SEQ IDs No. 1-4 or SEQ ID Nos. 7-17 binds.


SEQUENCE LISTING (19 sequences)

SEQ ID NO: 1 - DNA, 21 bp, Artificial Sequence
  primer
gatcctgccagtagtcatatg

SEQ ID NO: 2 - DNA, 21 bp, Artificial Sequence
  primer
ctatcctaccatcgaaagttg

SEQ ID NO: 3 - DNA, 20 bp, Artificial Sequence
  primer
ggttcattcaaatttctgcc

SEQ ID NO: 4 - DNA, 17 bp, Artificial Sequence
  primer
caccagacttgccctcc

SEQ ID NO: 5 - DNA, 20 bp, Artificial Sequence
  primer
attggagggcaagtctggtg

SEQ ID NO: 6 - DNA, 20 bp, Artificial Sequence
  primer
ccgatccctagtcggcatag

SEQ ID NO: 7 - DNA, 20 bp, Artificial Sequence
  primer
taggggatcgaagatgatca

SEQ ID NO: 8 - DNA, 19 bp, Artificial Sequence
  primer
gacctggtgagtttccccg

SEQ ID NO: 9 - DNA, 19 bp, Artificial Sequence
  primer
attgacggaagggcaccac

SEQ ID NO: 10 - DNA, 19 bp, Artificial Sequence
  primer
tgtacaaagggcagggacg

SEQ ID NO: 11 - DNA, 27 bp, Artificial Sequence
  hybridization probe
ggaccatcgtaatgattaatagggacg

SEQ ID NO: 12 - DNA, 23 bp, Artificial Sequence
  hybridization probe
cgtatgcccttcattgggtgtgc

SEQ ID NO: 13 - DNA, 22 bp, Artificial Sequence
  hybridization probe
tacctatggtgagtactgctgc

SEQ ID NO: 14 - DNA, 21 bp, Artificial Sequence
  hybridization probe
cgtccgcttaggcgagcactg

SEQ ID NO: 15 - DNA, 22 bp, Artificial Sequence
  hybridization probe
tacctatggtaagcactgttgc

SEQ ID NO: 16 - DNA, 22 bp, Artificial Sequence
  hybridization probe
acctgtactccttgtgggtgca

SEQ ID NO: 17 - DNA, 22 bp, Artificial Sequence
  hybridization probe
cgcttttttgcgagtactggac

SEQ ID NO: 18 - DNA, 26 bp, Artificial Sequence
  hybridization probe
agagtgttcaaagcaggcttkacgcc

SEQ ID NO: 19 - DNA, 30 bp, Artificial Sequence
  hybridization probe
aggccgtatgcccttcattgggtgtgcggt